(19) United States
(12) Patent Application Publication
(10) Pub. No.: US 2015/0128957 A1
(43) Pub. Date: May 14, 2015

(54) WARMING IN MEDICAL POSITIONERS

(71) Applicant: Eugene Lloyd Hiebert, Salem, OR (US)

(72) Inventor: Eugene Lloyd Hiebert, Salem, OR (US)

(21) Appl. No.: 14/542,378

(22) Filed: Nov. 14, 2014

Related U.S. Application Data
(60) Provisional application No. 61/904,333, filed on Nov. 14, 2013.
(60) Provisional application No. 61/909,243, filed on Nov. 26, 2013.

(51) Int. Cl. A61F 7/00; A61F 5/37; A61G 13/12

(52) U.S. Cl. CPC A61F 7/007; A61G 13/1275; A61F 5/3769; A61F 2007/0077
USPC 128/845; 128/870; 128/876

(57) ABSTRACT

Described herein are embodiments of vacuum activated evacuatable medical positioners, warming devices for use with such positioners, systems that include a positioner and a warming device, and related methods. The disclosed embodiments include an electrical warming medium that is positioned, or adapted to be positioned, external to the patient-bearing side of an air-tight shell of a positioner. The electrical warming medium may be an integral part of a positioner, a part of a circumferential sleeve adapted to be placed around a positioner, or part of a pad adapted to be placed over a positioner, for example.

### CROSS REFERENCE TO RELATED APPLICATION

This application claims the benefit of U.S. Provisional Application No. 61/904,333, filed Nov. 14, 2013, and U.S. Provisional Application No. 61/909,243, filed Nov. 26, 2013, which are incorporated by reference herein in their entirety.

### FIELD

This application is related to medical positioning devices and devices for providing heat to a patient during a medical, surgical, or diagnostic procedure.

### BACKGROUND

It can be important to keep a patient warm during various medical procedures, such as during surgery, imaging, etc. During some procedures, the patient is held in a desired position using a positioning device, such as a vacuum activated positioner that conforms to the patient's anatomy as air is evacuated to hold the patient in a desired position. Such a positioner can also include a warming device located inside of the evacuatable shell to warm a patient, as is described in U.S. Pat. No. 8,555,890 (“the '890 patent”), which is incorporated by reference herein in its entirety. However, such warming devices can be inaccessible inside the shell, must pass heat through a wall of the positioner shell to the patient, are integrally fixed to a particular positioner, include a power cord that passes through a seam in the evacuatable shell and can therefore cause leaks in the shell and thereby cause deactivation of the positioner, and/or can have other drawbacks.

### SUMMARY

Described herein are embodiments of vacuum activated evacuatable positioners, warming devices for use with such positioners, and systems that include a positioner and a warming device. The disclosed embodiments include an electrical warming medium that is positioned, or adapted to be positioned, external to the patient-bearing side of an air-tight shell of a positioner. The electrical warming medium may be an integral part of a positioner, a part of a circumferential sleeve adapted to be placed around a positioner, or part of a pad adapted to be placed over a positioner, for example.

An exemplary system includes a medical positioning device (i.e., a positioner) and an electrical warming medium. The positioner comprises a flexible shell defining a deflatable air-tight internal region. The shell has an outer surface configured for positioning a patient during a procedure and a plurality of beads are disposed within the internal region of the shell. The positioner is configured to engage lower and side portions of a patient upon evacuation of air from the internal region of the shell to hold the patient in a selected position during a procedure. The electrical warming medium is positioned outside of the shell and is configured to convert electrical current into heat for warming the patient while being held by the positioning device.

The electrical warming medium can be attached directly to the outer surface of the shell, such as with an adhesive or welding, and/or the electrical warming medium can positioned in a pocket between the outer surface of the shell and a superior membrane that overlies the electrical warming medium and the shell and is secured around its perimeter to the shell.

In some embodiments, the electrical warming medium is coupled to a circumferential sleeve that is placed around the positioner and is removable from the positioner. In such sleeves, the electrical warming medium can be positioned within a pocket of the sleeve or can be attached to one side of the sleeve with an opposite side of the medium exposed. In some embodiments, the sleeve can include grommets or other openings and the positioner can include corresponding openings so that one or more straps or other fasteners or connecting devices can be placed through the aligned openings, such as to secure a patient to the positioner.

In some embodiments, the electrical warming medium is coupled to a pad that is placed over a patient bearing surface of the positioner. The electrical warming medium can be positioned in a pocket of the pad or exposed on one side of the pad. The pad may or may not include openings that align with openings in a positioner so that straps or other fasteners or connecting devices can be placed through the openings, such as for securing a patient to the positioner.

The electrical warming medium can be coupled to a power cord that supplies electricity to the electrical warming medium, and the power cord can be located entirely outside of the air-tight internal region of the shell of an associated positioner, so that the cord does not pass through a hermetically sealed portion of the shell.

An exemplary method of positioning and warming a patient during a medical procedure includes (1) positioning a medical positioner and an electrical warming medium between a patient and a support surface with the patient being in a selected position for a procedure, the positioner comprising an air-tight shell and a plurality of beads within the shell, and the electrical warming medium being positioned outside of the shell between the patient and the shell; (2) evacuating air from the shell such that the positioner fittingly engages portions of the patient to hold the patient in the selected position; and (3) supplying electrical current to the electrical warming medium to warm the patient while the patient is being held by the positioner.

The foregoing and other objects, features, and advantages of the invention will become more apparent from the following detailed description, which proceeds with reference to the accompanying figures.

### BRIEF DESCRIPTION OF THE DRAWINGS

FIG. 1 is a top view of an exemplary positioner having a warming medium within a pocket on top of the positioner.

FIG. 2 is a cross-sectional end view of the position of FIG. 1.

FIG. 3 is a top view of another exemplary positioner having a warming medium on top of the positioner.

FIG. 4 is a perspective view of a sleeve for a positioner with a warming medium on an upper surface of the sleeve.

FIG. 5 is a perspective view of another sleeve for a positioner with a warming medium within a pocket on top of the sleeve.

FIG. 6 is a top view of a positioner with a sleeve positioned around the positioner, the sleeve having a warming medium on its upper surface. The positioner and sleeve include aligned grommets for restraining straps or other fasteners or connecting devices.

FIG. 7 is a top view of a positioner with a sleeve positioned around the positioner, the sleeve having a warming medium within a pocket. The positioner and sleeve include aligned grommets for restraining straps or other fasteners or connecting devices.

FIG. 8 is a cross-sectional end view of the positioner and sleeve of FIG. 7.

FIG. 9 is a top view of an alternative positioner with a corresponding sleeve positioned around the positioner, the sleeve having a warming medium on its upper surface.

FIG. 10A is a top view of the alternative positioner with another corresponding sleeve positioned around the positioner, the sleeve having a warming medium within a pocket on top of the sleeve.

FIG. 10B is a cross-sectional end view of the positioner and sleeve of FIG. 10A.

FIG. 11 is a perspective view of an exemplary pad having a warming medium within a pocket in the pad and also including grommets.

FIG. 12 is a top view of the pad of FIG. 11 positioned on top of a positioner. Straps pass through aligned grommets in the pad and the positioner.

FIG. 13 is a cross-sectional end view of the pad and positioner of FIG. 12.

FIG. 14 is a top view of a positioner and a pad having a warming medium on its upper surface placed on top of a positioner, and showing straps passing through aligned grommets in the pad and the positioner.

FIG. 15 is a cross-sectional end view of the pad and positioner of FIG. 14.

FIG. 16 is a top view of an alternative positioner and a corresponding pad having a warming medium on its upper surface placed on top of the positioner.

FIG. 17 is a top view of the alternative positioner and a corresponding pad having a warming medium within a pocket, the pad being placed on top of the positioner.

FIG. 18 is a cross-sectional end view of the pad and positioner of FIG. 17.

FIG. 19 is a cross-sectional end view of a patient held by an evacuated positioner supported by an underlying support surface, with one warming pad between the patient and the positioner, and another warming pad overlying the patient, and with fasteners securing the pads and the positioner together on the sides of the patient.

FIG. 20 is a top view of an animal patient positioned on a positioner with a warming pad between the animal and the positioner, with the animals legs restrained by straps passing through apertures in the pad and the positioner.

### DETAILED DESCRIPTION

Described herein are embodiments of vacuum activated evacuatable positioners and systems that include a positioner and a device for use with a positioners, which include a warming medium that is positioned outside of the patient-bearing side of the air-tight shell of the positioner. As used herein, the terms “positioner,” “positioning device” and “medical positioner device” mean any evacuatable positioner for using in holding a patient for any medical, surgical, diagnostic, and/or imaging procedure. The term “medical procedure” is used broadly to include any medical, surgical, diagnostic, imaging, or related procedure. A warming medium can comprise any electrically conductive medium such as, but not limited to, conductive fabric, conductive mesh, conductive wires, fabric with a conductive coating, fabric with conductive wires, mylar conductive membrane, other conductive membranes, a membrane with printed circuitry, or other conductive heat producing media. In disclosed embodiments, a warming medium is coupled to or positioned over the upper side of the patient surface of the shell in an area size which is commensurate with, or corresponds to, the area size of the patient bearing surface of the shell. The disclosed warming technology can be used in positioners for humans and animals. The word “patient” includes humans, animals or any other non-animal subjects.

In some embodiments, a layer, film or membrane, such a layer of similar material as the patient bearing surface of the shell, or other functionally acceptable material, can be affixed over the warming medium on the patient bearing surface and sealed around the perimeter of the warming medium so that the warming medium is in a sealed pocket outside of the patient-bearing side of the air-tight shell of the positioner. In some embodiments, the warming medium is adhered to the interior inferior and the interior superior surfaces of such a sealed pocket with appropriate adhesives so that an electrical short does not occur when the surface of the warming medium comes in close contact with itself, as when the surface is wrinkled or folded over on itself. A cable conducting electrical power to the warming medium can exit from this pocket in a sealed fashion without passing through the air-tight shell of the positioner.

In some embodiments, the warming medium is attached to the upper surface of the positioner but is not covered by a superior membrane, such that the warming medium forms the upper-most surface on the patient bearing side of the positioner. The warming medium can be adhered to the outer surface of the air-tight shell with an adhesive, welding, mechanical fasteners, or other suitable securement means. A cable conducting electrical power to the warming medium can extend from the warming medium without passing through the air-tight shell of the positioner.

In some embodiments, the warming medium can be included in or on a circumferential sleeve or slip cover that is configured to be placed around and/or slid over a positioner. In some embodiments, the warming medium can be in a pocket formed in the sleeve or slipcover, or can be exposed on an upper or inner surface of the sleeve or slip cover. In such embodiments, the sleeve or slip cover can be readily removed from a positioner and placed back around a same or different positioner. This also allows for retrofitting of existing positioners to add patient warming functionality.

In still other embodiments, the warming medium can be included in a pad or insert that is configured to be placed on top of a positioner, between the positioner upper bearing surface and a patient. Such embodiments can be similar to a sleeve or slipcover, without extending all the way around the positioner. Instead, such embodiments may be secured to a positioner using straps or other fasteners or connecting devices passing through aligned grommets and/or in other manners.

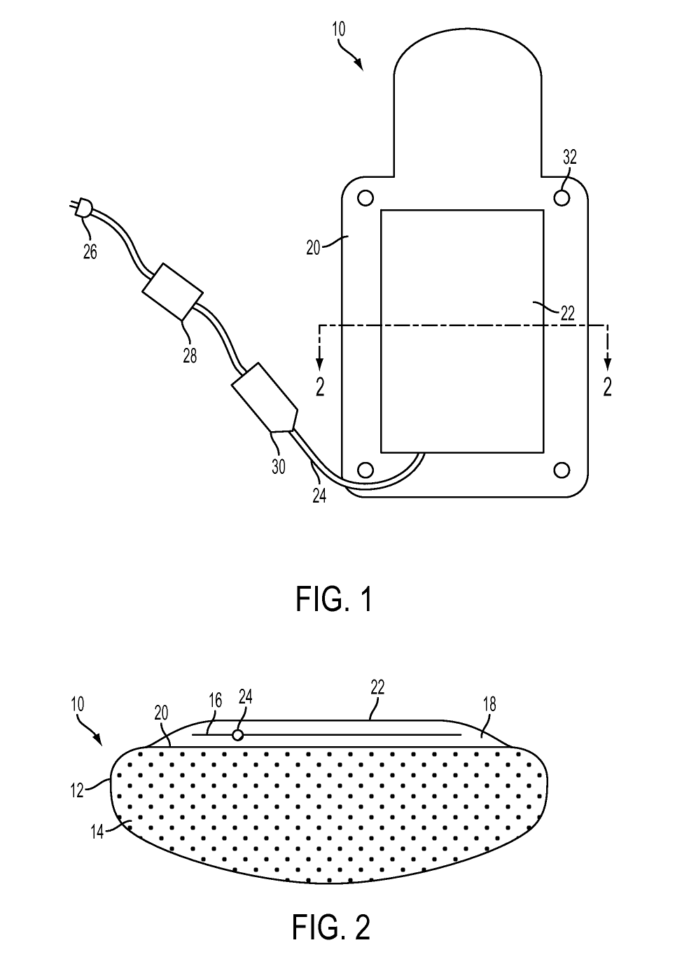

FIGS. 1 and 2 show an exemplary positioner 10 having an air-tight shell 12 with internal beads 14 and a warming medium 16 positioned in a sealed pocket 18 formed between the upper side or patient bearing surface 20 of the shell 12 and a superior layer 22. The warming medium 16 is electrically coupled to a power cord 24 that exits the pocket 18 outside of the air-tight shell 12. The power cord 24 can include a plug 26 for an AC outlet, an AC to DC converter 28, a heat level controller 30, and/or other features. The warming medium 16 can be secured to the upper side 20 of the shell and/or to the underside of the superior layer 22, such as using an electrically insulating adhesive, to keep the warming medium from folding or bunching and creating a short circuit. In other embodiments, the warming medium 16 can be held within the pocket by other means, such as welding, or can be free-floating within the pocket.

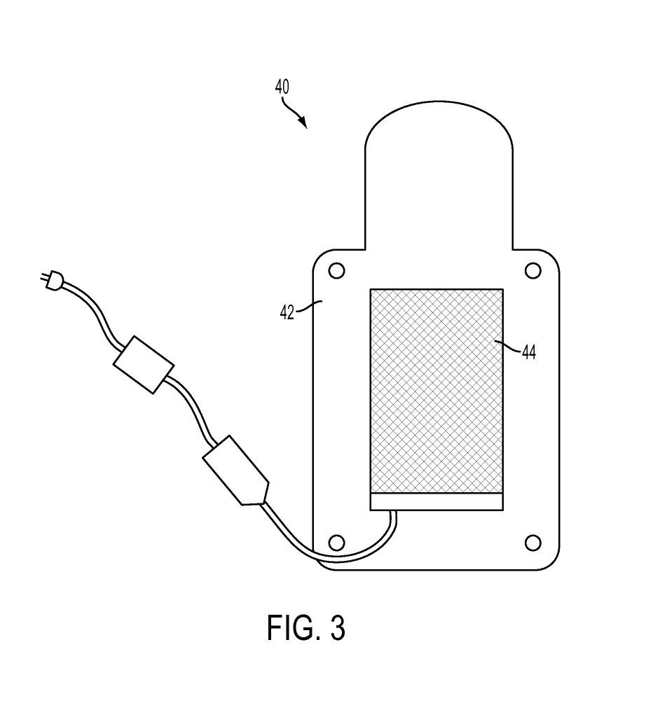

FIG. 3 shows another exemplary positioner 40, similar to positioner 10, but with an warming medium 44 coupled to the upper side or patient bearing surface 42 of the positioner without a superior layer of material sealing it in a pocket.

The positioners shown in FIGS. 2 and 3 can have any size and shape, and are not limited to the particular shaped positioners shown.

Many vacuum activated positioners for humans and animals exist which have no patient warming technology and which cannot be remanufactured to contain patient warming technology. With the technology described herein, such positioners currently without patient warming technology can be retrofitted to have patient warming technology.

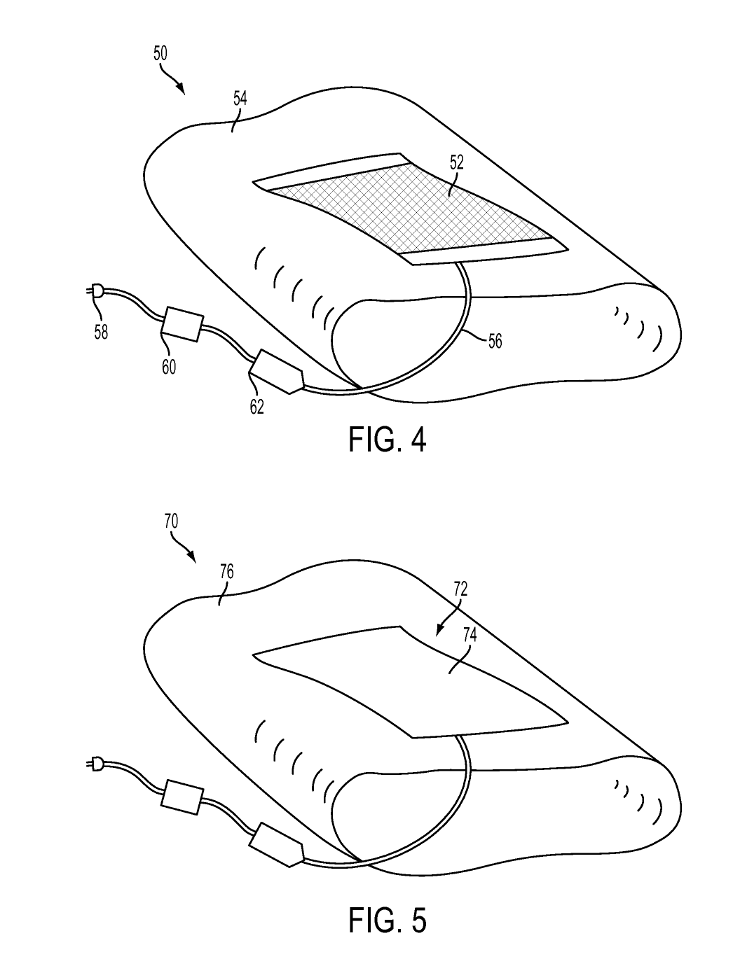

Exemplary circumferential sleeves (also referred to as slipcovers) disclosed herein (e.g., FIGS. 4-10) can fit around the longitudinal axis, or other axis, of a particular positioner and can be configured to fit the unique size, shape and accoutrements of each particular positioner. An exemplary sleeve can comprise the same or similar material as a positioner or other functionally acceptable material. A warming medium (commensurate in surface area to the patient bearing surface of the positioner, or having other size) can be enclosed in a sealed pocket on the patient bearing surface of a sleeve or can be attached to the patient bearing surface without a superior layer. A power input cable, AC to DC adaptor, and/or heat controller can be included.

FIG. 4 depicts an exemplary circumferential sleeve 50 with a warming medium 52 coupled to the patient bearing surface 54 along with a power cable 56, plug 58, AC to DC converter 60, and a heat controller 62. FIG. 5 shows a circumferential sleeve 70 with a sealed pocket 72, which contains a warming medium 75 (FIG. 8). The pocket 72 is formed by superior membrane or layer 74 sealed around its perimeter to the upper surface 76 of the sleeve. The sleeves 50, 70 can have any size and can be comprised of any material that allows it to be retained around a positioner. They may be made of the same material as the positioner or other material that is acceptable for use with the warming medium. A circumferential sleeve can be made of sufficient size, length and circumference to accommodate the positioner it envelops and the area of the warming medium necessary for warming of the patient that is borne by that positioner. In some embodiments, the sleeve can comprise an elastic material that stretches and conforms snuggly around a positioner. In some embodiments, the sleeve can be separated longitudinally to allow it to be wrapped around a positioner and then the ends can be reattached, such as with Velcro or other mechanisms, to regain the circumferential shape and secure the sleeve around the positioner.

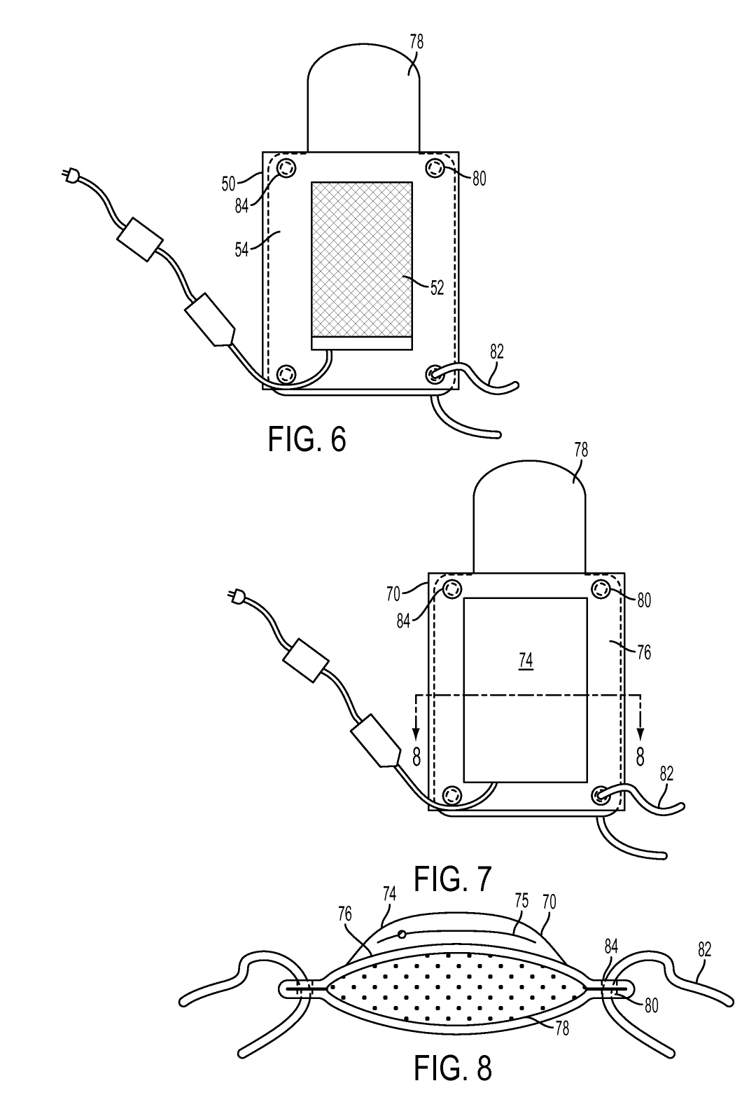

FIG. 6 shows the circumferential sleeve 50 positioned around an exemplary positioner 78, and FIG. 7 shows the sleeve 70 positioned around the positioner 78. FIG. 8 is a cross-sectional view of the sleeve 70 positioned around the positioner 78. Portions of the positioner 78 may extend outside of ends of the sleeve. Like the illustrated positioner 78, some positioners have grommet openings 80, such as at four corners of the positioner, to accommodate restraint straps 82 or other fasteners or connecting devices. The circumferential sleeves 50, 70 can also have openings 84 at corresponding locations to also accommodate the restraint straps 82 and thereby also prevent longitudinal, lateral and circumferential slippage of the sleeve when the patient is placed on the patient bearing surface of the sleeve or when the positioner is manipulated and/or vacuum activated for use.

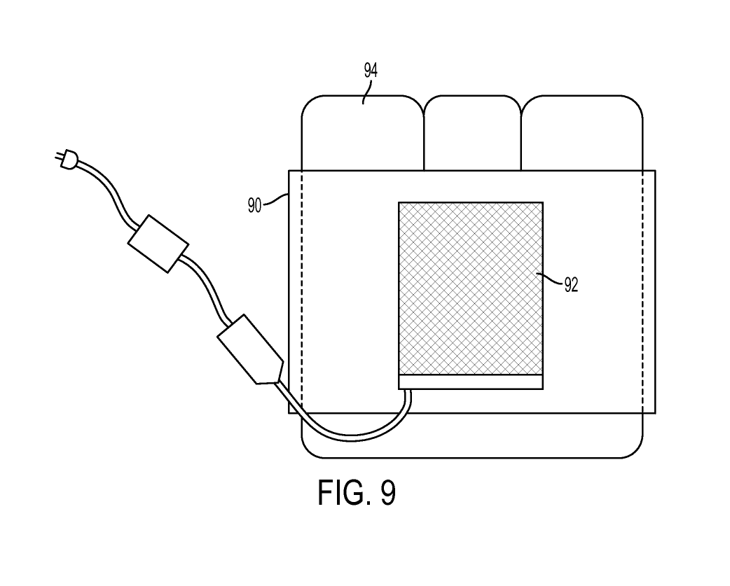

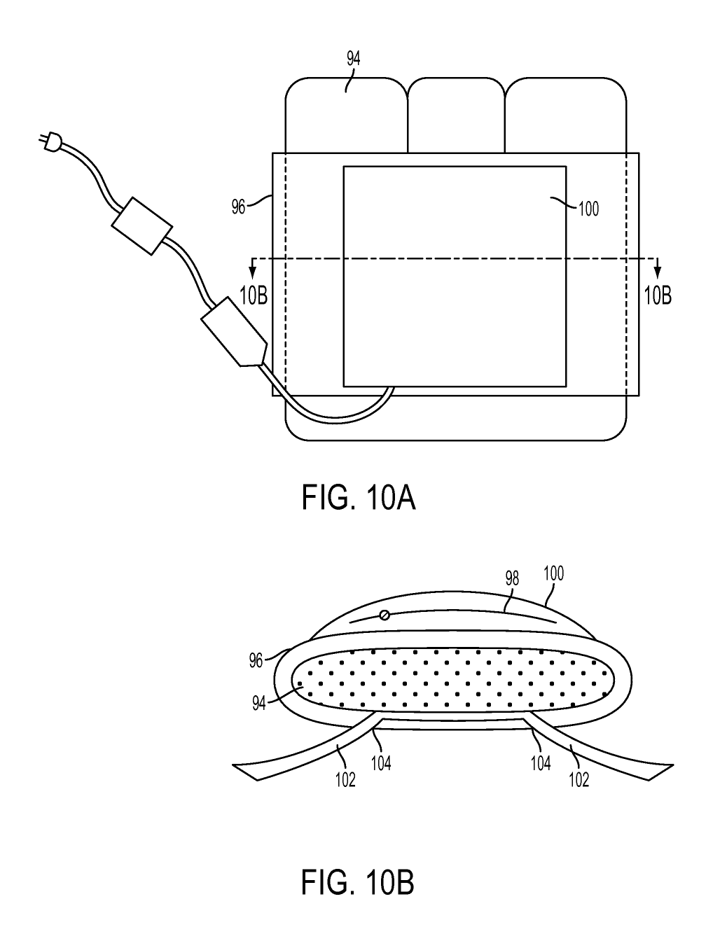

As shown in FIGS. 9, 10A, and 10B, for example, a circumferential sleeve can be placed around other types of positioners. FIG. 9 shows a sleeve 90 with an uncovered warming medium 92 on its upper surface positioned around an alternative positioner 94. FIGS. 10A and 10B shows a sleeve 96 with the warming medium 98 in a sealed pocket between patient bearing surface of the sleeve and a superior layer 100. FIG. 10B is a cross-section showing fastening straps 102 on the underside of a positioner 94 projecting through commensurately located holes 104 in the bottom of the sleeve 96 for anchoring the positioner to an underlying support surface, such as a surgical table, etc. Besides allowing the positioner 94 to be fastened with its straps as intended, the projection of the straps 102 through the openings 104 in the sleeve 96 can also prevent the sleeve from shifting circumferentially, longitudinally and/or laterally when in use.

These illustrations do not limit the types, sizes and configurations of positioners which can be utilized with such circumferential warming sleeves.

In the described sleeves, the warming medium is electrically coupled to an electrical power cord that supplies electricity to the warming medium. The power cord can include an AC outlet plug, an AC to DC electrical converter, a heat level controller, and/or other features. The disclosed sleeves can be used for both human and animal positioners or patient bearing surfaces.

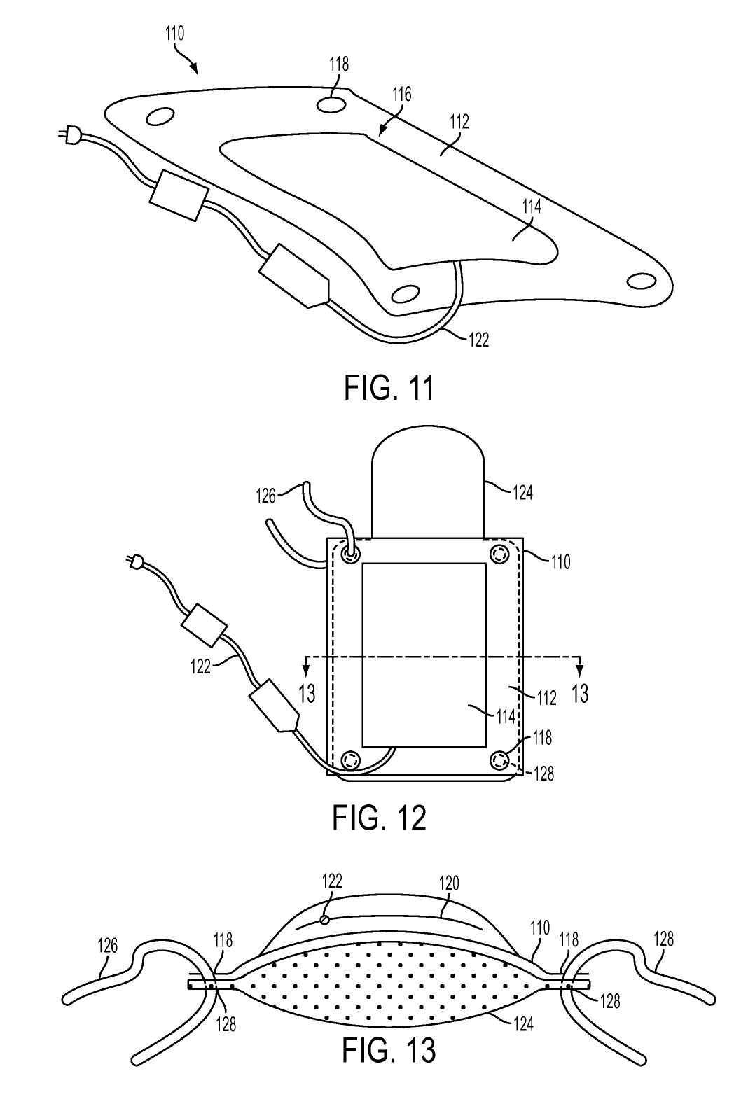

In other embodiments, a warming medium can be included in a pad (also referred to as an insert) that can be placed on the patient bearing surface of a positioner and/or against other surfaces of a patient. FIG. 11 shows an exemplary warming pad 110 that comprise an inferior layer 112 (the underside of which can be placed against the patient bearing surface of the positioner), a superior layer 114 attached to the upper surface of the inferior layer around its perimeter to form a pocket 116, and a warming medium 120 (FIG. 13) positioned in the pocket. The pad 110 can also include grommets or other openings 118 in the inferior layer 112 to help secure the pad to a positioner, as shown in FIGS. 12 and 13.

FIGS. 12 and 13 show the pad 110 overlying an exemplary positioner 124 with the grommets 118 in the pad aligned with grommets 128 in the positioner and with straps 126 passing through both openings. The straps 126, or other fasteners or connecting devices, can be used for restraining a patient to the positioner and can also keep the pad 110 aligned between the patient and the positioner by restricting lateral and/or longitudinal shifting of the pad.

The inferior and superior layers 112, 114 can be comprised of any functionally compatible material which can be sealed so that the internal electrical medium and circuitry is protected within the pocket 116. The warming pad can have other appurtenances for attachment to the underlying positioner or may have no appurtenances for attachment at all (as in the embodiments shown in FIGS. 18-20).

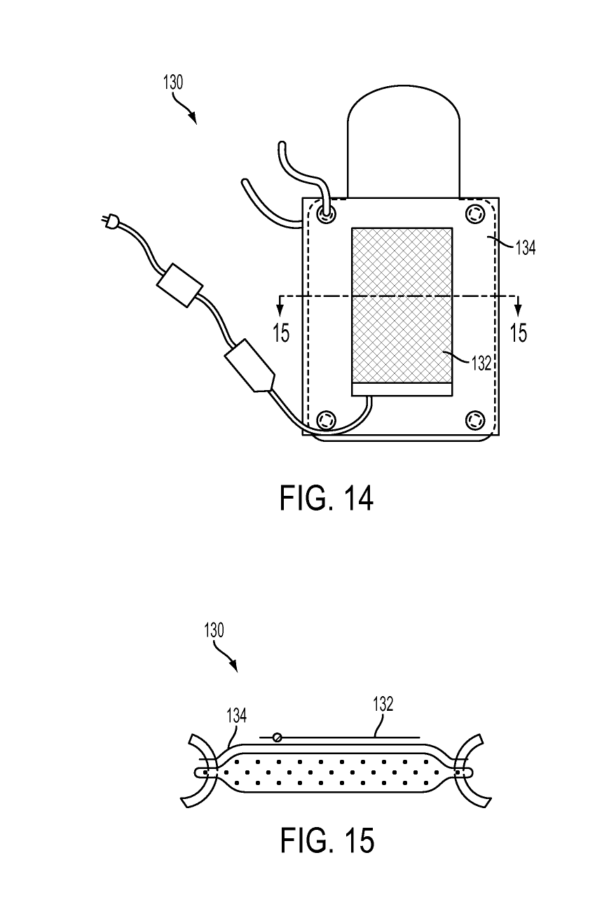

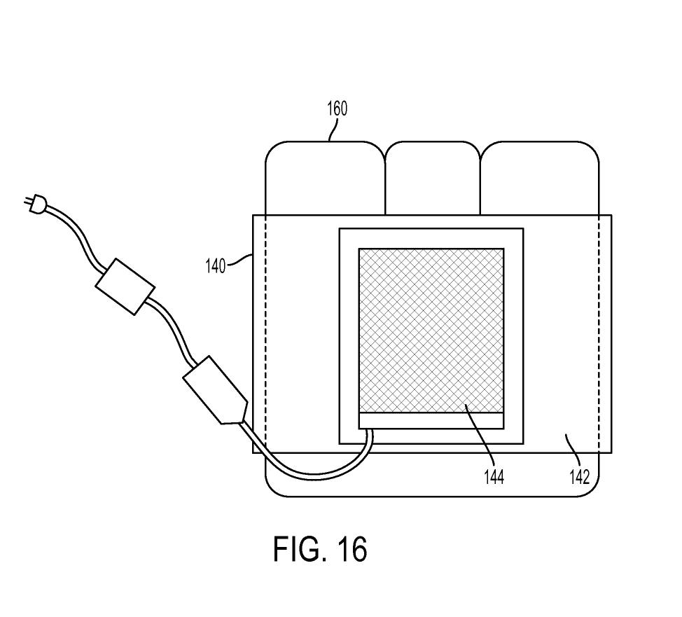

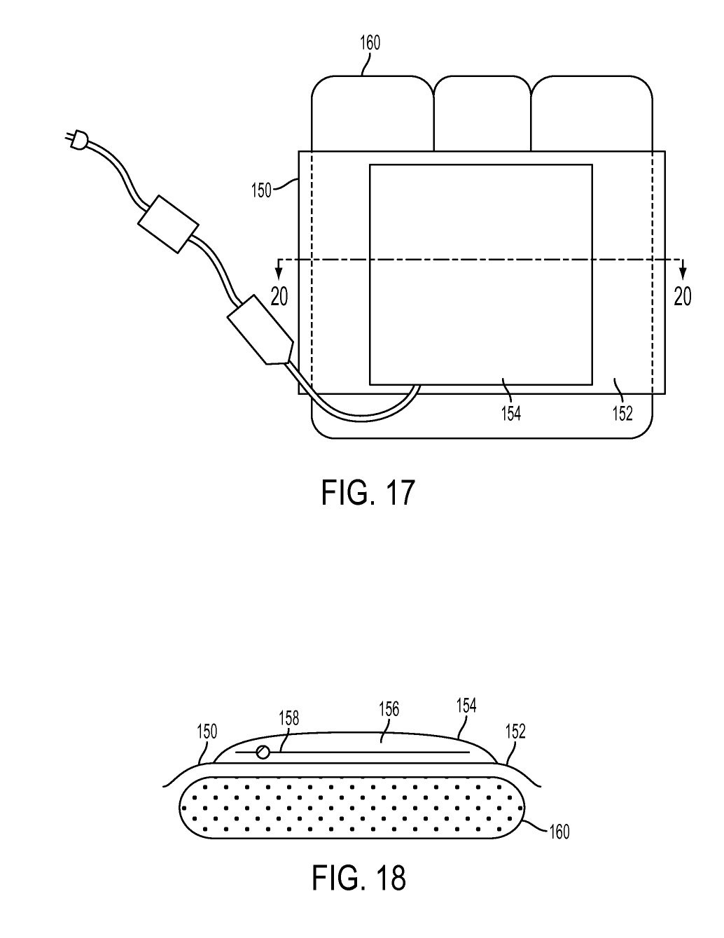

FIGS. 14 and 15 show an alternative pad 130 that is similar in all respects to the pad 110, except that no superior layer or pocket is included and the warming medium 132 is secured directly to the inferior layer 134 (e.g., via an adhesive or welding) with the upper side of the warming medium exposed.

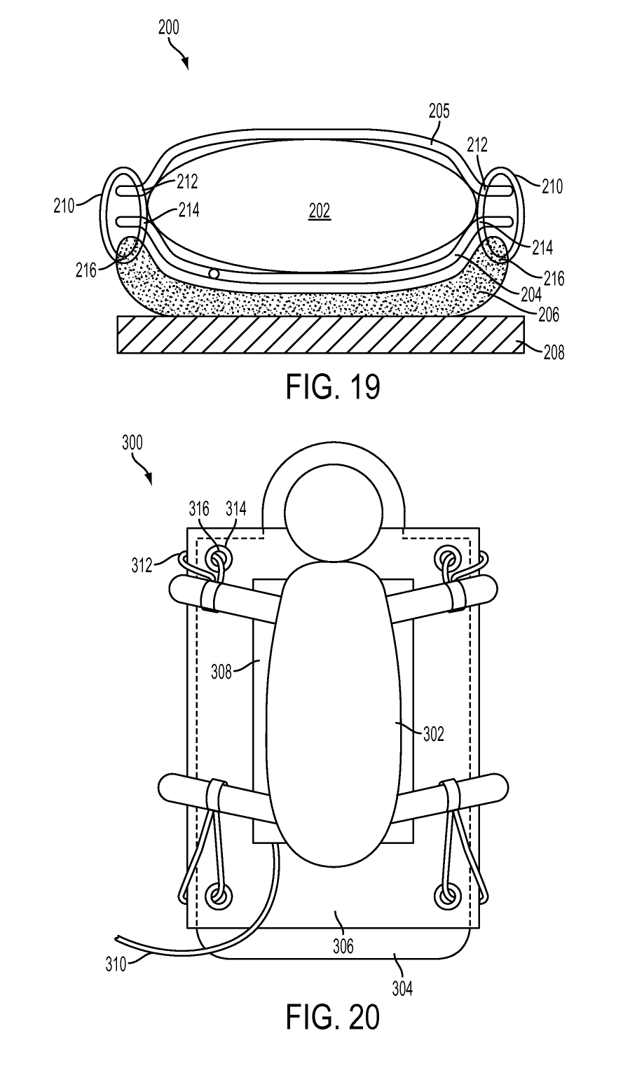

FIGS. 18-20 show alternative pads 140 and 150 configured to overly any size positioner without direct attachment of the pad to the positioner. An alternative, larger sized positioner 160 is shown. FIG. 18 shows pad 140 which includes an inferior layer 142 with an electrical warming medium secured to the top of the inferior layer. FIGS. 19 and 20 show pad 150 which includes an inferior layer 152 and a superior layer 154 secured to the inferior layer around its perimeter to form a pocket 156 that contains the electrical warming medium 158. Because the pads 140, 150 simply overly the positioner, without needing grommets or other attachments to the positioner, the same pads can be used with many different sized positioners. The weight of a patient and/or the conformation of the positioner to the anatomy of the patient can hold the pad in place against the top of the positioner. The pads 140, 150, and other pads disclosed herein, can also be used apart from a positioner, such as by placing a pad on top of a patient while the patient is held on the lower side by a positioner.

In the described pads, the warming medium is electrically coupled to an electrical power cord that supplies electricity to the warming medium. The power cord can include an AC outlet plug, an AC to DC electrical converter, a heat level controller, and/or other features. The disclosed pads can be used for both human and animal positioners or patient bearing surfaces.

In any of the described embodiments, the electrical warming medium can be configured to be powered by one or more portable power sources, such a DC battery. This can be instead of, or in addition to, being able to be plug into and be powered by a AC power outlet. The capability of being powered by a portable power source can enable the positioner, sleeve, pad, etc. to be used remotely and/or mobily, such as by a first responder, in an ambulance, or during a power outage.

FIG. 19 is a cross-sectional end view of an exemplary arrangement 200 wherein a patient 202 is held by an evacuated positioner 206 supported by an underlying rigid support surface 208, with one warming pad 204 between the patient and the positioner, and another warming pad 205 overlying the patient, and with fasteners 210 securing the pads and the positioner together on the sides of the patient. The pads 204, 205 contain an electrical warming medium and can be individually coupled to a power source. The fasteners 210 can pass through openings 212, 214, and 216 in the top pad 205, the bottom pad 204, and the positioner 206, respectively. The fasteners 210 can comprise straps, clips, or any other type of fasteners or connecting device. In some arrangement, the top and bottom pads 204, 205 can be secured together independent of the positioner 206. In some arrangements, the positioner 206 and/or the pads 204, 205 can be secured to the support surface 208.

In an exemplary method, after a medical/surgical/diagnostic procedure is performed on the patient, the patient along with a bottom warming pad and/or a top warming pad can be lifted off of/out of a positioner, with the warming pads remaining in contact with the patient, and moved to a second location, such a post-operative or recovery area, with the warming pads remaining in place to keep the patient warm. This can be particularly beneficial with patients prone to losing body heat rapidly, such as animals with high metabolism, low body fat, etc., patients with hypothermia, etc.

FIG. 20 is a top view of an arrangement with an animal patient 302 positioned on a positioner 304 with a warming pad 306 between the animal and the positioner, with the animals legs restrained by straps 312 passing through apertures 314, 316 in the pad and the positioner. The warming pad 306 includes a pocket 308 that contains an electrical warming medium coupled to a power source via cord 310.

In any of the describe arrangements, the sleeves, slipcovers, pads, or inserts, can be configured to partially surround the positioner. For example, the device can include an elastic band around its perimeter that is configured to wrap around the sides of the positioned and be positioned along the bottom of the positioner, akin to a fitted bed sheet. In other embodiments, straps, clips, or other fasteners can retain the edges of the warming device to the sides and/or bottom of the positioner, or can connect on side the warming device to the other side of the positioner device below the positioner.

The disclosed technology can provide any combination of the following enumerated benefits and advantages, in addition to what has been described elsewhere herein:

1. Disclosed embodiments can avoid the necessity of having a hermetic seal around the power conduction cable as it exits through the shell from the interior of the positioner. Typical positioners are vacuum activated and, therefore, may require hermetic seals of all exiting members from the interior of the positioner so that the vacuum can be maintained. With time and wear on the positioner, a hermetic seal around a cable exiting from the interior of the positioner may be broken resulting in ineffective use of the positioner.

2. The interior of a vacuum activated positioner has many (e.g., millions) flexible and/or polystyrene beads which become amassed when the positioner is activated with suction or vacuum. With the warming medium coupled to the underside of the patient bearing surface inside the shell, consequent use and handling can induce the internal beads and pieces thereof to work their way through the warming medium and become lodged between the warming medium and the underside of the patient bearing surface. This can cause insulation and a reduction of heat transmittance to occur from the warming medium to the patient. When the warming technology is located on the outer side of the patient bearing surface of the shell, and not in the interior of the positioner, this potential insulation and reduction of heat transmittance can be avoided.

3. The warming medium, in some embodiments, has one or more busbars affixed to its opposing edges for conduction of electricity from the power source to the electrically conductive or resistive warming medium. If coupled to the underside of the patient bearing surface inside the shell, there may not be an undergirding supportive membrane or film to the warming medium. With the warming technology positioned adjacent the upper side of the patient bearing surface of the shell there can be a supportive layer or membrane of the positioner shell material on the underside of the warming medium. This underside support to the busbars and conductive elements may prevent premature breakage of these members with use and wear and tear of the positioner. Some embodiments also include upper side membranes to further support the busbars and conductive elements.

4. By coupling of the warming medium to the outer side of the shell, the warming medium can be secured to the shell using minimal or no thermo-conductive adhesives.

5. Positioning the warming medium above the upper side of the patient bearing surface of the shell avoids the placement of the warming medium within the hermetically sealed interior of the positioner. If breakage of wires or busbars on the warming medium occurs with use and wear and tear, repair of these electrical conductors or replacement of the entire warming medium can be performed without entering the interior of the positioner. This avoids violation of the hermetic seal and spillage of beads from the interior of the positioner. Repair and maintenance can therefore be expedited and/or more easily facilitated since a hermetic seal is functionally not required.

6. Coupling the warming medium to the upper side of the patient bearing surface of the positioner can simplify manufacturing. Affixing a membrane or layer of similar material over the warming medium can be accomplished by adhesives, radio-frequency welding, or other sealing methods around the perimeter of the warming medium to make a sealed pocket. The sealing of the exiting power cable from the pocket can be accomplished with adhesive, radio-frequency or sealing technology.

7. Disclosed sleeves, slipcovers, inserts or pads can have the advantage of being usable for warming patients in situations where the patient positioning device or underlying surface has no independent source of patient warming.

8. An existing patient positioner or patient bearing surface can be retrofitted for patient warming using the disclosed technology. Disclosed sleeves, slipcovers, inserts or pads can be easily placed around or on a patient positioner or patient bearing surface prior to placement of the patient onto the patient bearing surface and can be secured in place or not secured in place as required for the clinical situation.

9. The size of a warming insert or pad need not correspond to the size or shape of the positioner or patient bearing surface which it overlies. For example, it may overhang the underlying positioner or be significantly smaller than the underlying positioner support surface, but the insert or pad can provide warmth to the patient even though the fit may not be exact. Thus, any given size insert/pad can be used with many different sized positioners, which avoids having to stock specific sized warming devices for each specific type and size of positioner.

10. A warming insert or pad can also be placed over a patient instead of and/or in addition to beneath the patient on the patient bearing surface for improved patient warming.

11. Two warming inserts/pads can be used together to provide greater body warming, such as for anesthetized small animals, which may have a larger surface area to mass ratio and a higher metabolic rate which creates greater body heat loss. In such a circumstance, a warming insert/pad can be placed on the superior surface of a positioner, the patient can then be placed on the superior surface of the warming insert/pad, and another warming insert/pad can then be placed over the superior surface of the patient so that the patient is now surrounded inferiorly, superiorly and possibly laterally by warming surfaces.

12. Because the underlying positioner (e.g., a HUG-U-VAC Veterinary positioner) can act as an insulator against heat loss, in some situations a warming insert/pad need not be placed on the superior surface of the positioner underneath the patient. A warming insert/pad can instead be placed over the superior surface of the patient which provides sufficient warming of the patient.

13. The disclosed warming pads can allow for extra cushioning between the positioner and the patient. For example, closed cell foam or other cushioning material can be placed on the superior surface of the positioner with the warming insert/pad being placed on the superior surface of the cushioning material and the patient placed on the superior surface of the warming pad. This arrangement allows for extra cushioning in special positioning circumstances without interference with patient warming.

14. Where a patient is laying on the warming insert/pad, while in the positioner, the insert/pad can be used to lift the patient out of or off of the positioner, can be used to carry the patient to a postoperative area, and/or can then be used to also provide for body warming during a postoperative period.

15. The use of an external device, such as a pad or sleeve, can greatly increase the ability to clean and maintenance the warming device, the associated pad or sleeve, as well as the positioner itself.

Any of the disclosed embodiments may alternatively be used with, or include instead, a non-evacuatable patient positioner in an analogous manner.

For purposes of this description, certain aspects, advantages, and novel features of the embodiments of this disclosure are described herein. The disclosed apparatuses, systems, and methods should not be construed as limiting in any way. Instead, the present disclosure is directed toward all novel and nonobvious features and aspects of the various disclosed embodiments, alone and in various combinations and sub-combinations with one another. The disclosed embodiments are not limited to any specific aspect or feature or combination thereof, nor do the disclosed embodiments require that any one or more specific advantages be present or problems be solved.

As used herein, the term “and/or” used between the last two of a list of elements means any one or more of the listed elements. For example, the phrase “A, B, and/or C” means “A,” “B,” “C,” “A and B,” “A and C,” “B and C” or “A, B and C.” As used herein, the term “coupled” generally means mechanically, chemically, electrically, magnetically or otherwise coupled or linked and does not exclude the presence of intermediate elements between the coupled items, unless otherwise described herein.

In view of the many possible embodiments to which the principles disclosed herein may be applied, it should be recognized that the illustrated embodiments are only examples and should not be taken as limiting the scope of the disclosure. Rather, the scope of the disclosure is at least as broad as the following claims. I therefore claim all that comes within the scope and spirit of these claims.

1. A system comprising:
a medical positioning device having a flexible shell defining a deflatable air-tight internal region, the shell comprising an outer surface configured for positioning a patient during a procedure, and a plurality of beads disposed within the internal region of the shell, wherein the medical positioning device is configured to engage lower and side portions of a patient upon evacuation of air from the internal region of the shell to hold the patient in a selected position during a procedure; and
an electrical warming medium coupled to the medical positioning device and positioned outside of the shell, the electrical warming medium being configured to convert electrical current into heat for warming the patient while being held by the positioning device.

2. The system of claim 1, wherein the electrical warming medium is attached to the outer surface of the shell.

3. The system of claim 1, wherein the electrical warming medium is positioned in a pocket that is located above the outer surface of the shell and below a superior layer that overlies the electrical warming medium.

4. The system of claim 1, wherein the electrical warming medium is coupled to a sleeve that is placed around the medical positioning device and is removable from the medical positioning device.

5. The system of claim 4, wherein the electrical warming medium is positioned within a pocket of the sleeve.

6. The system of claim 4, wherein the sleeve includes one or more sleeve openings and the medical positioning device includes one or more positioner openings, and the system includes one or more fasteners or connecting devices that extend through the one or more sleeve openings and through the one or more positioner openings.

7. The system of claim 6, wherein the one or more fasteners or connecting devices are configured to secure a patient to the medical positioning device and to the sleeve.

8. The system of claim 1, wherein the electrical warming medium is coupled to a pad that is placed over a patient bearing surface of the medical positioning device.

9. The system of claim 8, wherein the electrical warming medium is positioned in a pocket of the pad.

10. The system of claim 8, wherein the pad includes one or more pad openings and the medical positioning device includes one or more positioner openings, and the system includes one or more fasteners or connecting devices that extend through the one or more pad openings and through the one or more positioner openings.

11. The system of claim 10, wherein the one or more fasteners or connecting devices are configured to secure a patient to the medical positioning device and to the pad.

12. The system of claim 8, wherein the system includes a second pad that includes a second electrical warming medium and that is adapted to be placed on a surface of the patient that is not in contact with the medical positioning device.

13. The system of claim 1, wherein the electrical warming medium is coupled to a power cord that supplies electricity to the electrical warming medium, and the power cord is located entirely outside of the air-tight internal region of the shell.

14. The system of claim 3, wherein the electrical warming medium is secured to the overlying superior layer or an underlying layer with an electrically insulating adhesive to prevent the electrical warming medium from folding against itself and short circuiting.

15. A warming device adapted to be used with a medical positioning device that includes plurality of beads disposed within the internal region of the medical positioning device and that conforms to a patient's anatomy when the internal region is evacuated of air, the warming device comprising an electrical warming medium and a power cord for supplying electrical current to the electrical warming medium, the warming device further comprising an attachment feature for securing the warming device to the medical positioning device with the electrical warming medium located outside of the internal region and adjacent to an external patient bearing surface of the medical positioning device, wherein the warming device is configured to provide heat to a patient while the patient is being held by the medical positioning device.

16. The warming device of claim 15, wherein the attachment feature comprises one or more grommets configured to receive straps, fasteners or connecting devices for restraining a patient to the medical positioning device.

17. The warming device of claim 15, wherein the attachment feature comprises a circumferential sleeve configured to extend around the medical positioning device.

18. The warming device of claim 15, wherein the warming device includes a pocket formed between an inferior layer and a superior layer, the electrical warming medium is positioned within the pocket, the inferior layer includes the attachment feature for securing the warming device to the medical positioning device, and the superior layer is adapted for conducting heat from the electrical warming medium to the patient being held by the medical positioning device.

19. A method of positioning and warming a patient during a procedure, the method comprising:
positioning an electrical warming medium adjacent an external patient engaging surface of an air-tight evacuatable shell of a medical positioning device;
positioning the medical positioning device and the electrical warming medium between a patient and a support surface with the patient being in a selected position for a medical, surgical, or diagnostic procedure;
evacuating air from the shell such that the positioner fittingly engages portions of the patient to hold the patient in the selected position; and
supplying electrical current to the electrical warming medium to warm the patient while the patient is being held by the medical positioning device.

20. The method of claim 19, wherein the electrical warming medium is part of a pad that is separate from the medical positioning device, and the method further comprises placing one or more straps, fasteners or connecting devices through openings in the pad to hold the pad on a patient bearing side of the medical positioning device.

21. The method of claim 19, wherein the electrical warming medium is part of a sleeve that is separate from the medical positioning device, and the method further comprises placing the sleeve around the medical positioning device such that the electrical warming medium is adjacent to a patient bearing surface of the medical positioning device.

22. The method of claim 19, further comprising:
after the procedure, separating the patient and the electrical warming medium from the medical positioning device with the electrical warming medium remaining in contact with, or adjacent to, the patient;
transporting the patient and the electrical warming medium from a location of the medical positioning device to a second location, with the electrical warming medium remaining in contact with, or adjacent to, the patient; and
continuing to warm the patient with the electrical warming medium at the second location.
